(19) United States
(12) Patent Application Publication
(10) Pub. No.: US 2026/0242822 A1
(43) Pub. Date: Aug. 20, 2026

(54) METHODS AND KITS FOR THE IMPROVED FERMENTATIVE PRODUCTION OF A RECOMBINANT VIRUS

(71) Applicant: Takeda Pharmaceutical Company Limited, Osaka (JP)

(72) Inventors: Juan Antonio Hernandez Bort, Orth an der Donau (AT); Carolin Isabel Kahlig, Orth an der Donau (AT); Lucia Micutkova, Orth an der Donau (AT); Barbara Krauss, Orth an der Donau (AT); Johannes Grillari, Wien (AT)

(73) Assignee: Takeda Pharmaceutical Company Limited, Osaka (JP)

(21) Appl. No.: 18/874,506

(22) PCT Filed: Jun. 27, 2023
(86) PCT No.: PCT/IB2023/056597

§ 371 Date: Dec. 12, 2024

(30) Foreign Application Priority Data
Jun. 27, 2022 (EP) 22181387.6

(51) Int. Cl. C12N 15/86

(52) U.S. Cl. CPC C12N 15/86; C12N 2740/16043; C12N 2740/16051; C12N 2750/14143; C12N 2750/14151

(57) ABSTRACT

The present invention relates to methods and kits for the improved fermentative production of recombinant adeno-associated virus (AAV), adenovirus, lentivirus or retrovirus such as gammaretrovirus in HEK293 cells. Further, uses for increasing the fermentative production of said viruses in HEK293 cells are described.

### TECHNICAL FIELD OF THE INVENTION

The present invention relates to methods and kits for the improved fermentative production of recombinant adeno-associated virus (rAAV), adenovirus, lentivirus or retrovirus such as gammaretrovirus in HEK293 cells. Further, uses for increasing the fermentative production of said viruses in HEK293 cells are described.

### BACKGROUND OF THE INVENTION

The field of gene therapy with recombinant adeno-associated viruses (rAAV) has gained maturity since the first clinical trials on cystic fibrosis about 18 years ago. rAAV possesses interesting attributes for gene therapy. Although rAAV can induce a transient increase in cytokine level upon injection in mice, it was found to induce minimal immunogenicity and little to no inflammation hours later. rAAV is suitable for many medical applications as it can transduce non-dividing cells in various tissues or organs. Clinical evidence following gene therapy treatment with rAAV for lipoprotein lipase deficiency (alipogene tiparvovec or Glybera®) and liver congenital amaurosis also indicates that rAAV is well tolerated and that it can drive transgene expression for years in humans.

Mammalian cell production systems are used for rAAV production. The cells can support replication of viral vectors (e.g. recombinant adenoviruses, AdV).

Transient transfection methods with two or three plasmids have also been developed to produce rAAV. These methods can be used for testing an rAAV construct quickly but also, they can be scaled-up to 1000 L. An advantage of transient transfection is the absence of residual helper virus contaminants. Some stable cell lines (producer/packaging cells) have also been generated to facilitate rAAV production.

HEK293 cell lines have been used for rAAV production. They can be easily adapted to suspension culture in serum-free medium. Furthermore, numerous viral vectors have been already approved for phase I/II/III clinical trials. Originally, HEK293 cells are grown in adherence limiting scalability for the production of rAAV. This limitation has been overcome by adaptation of the HEK293 cell line to grow in suspension.

While efficient large-scale production of rAAV could be achieved in special genetically engineered HEK293 such as HEK293T cells with inserted SV40 large T antigen or with proprietary high producer HEK293 cells, there was a need for an rAAV production system based on HEK293 cells which does not require a genetic modification of the HEK293 cell and does not require the use of proprietary high producer cells.

### SUMMARY OF THE INVENTION

In a first aspect, the present invention provides a method for the fermentative production of a recombinant adeno-associated virus (rAAV), an adenovirus, a lentivirus or a retrovirus comprising the steps of:
Step 1: culturing the rAAV, the lentivirus, the adenovirus or the retrovirus in a HEK293 cell in a culture medium comprising an amount of an interferon (IFN) inhibitor over an incubation period; and
Step 2: recovering the rAAV, the lentivirus, the adenovirus or the retrovirus from the cell culture.

In a second aspect, the present invention provides a method for the fermentative production of an adeno-associated virus (AAV), a lentivirus, an adenovirus or a retrovirus comprising the steps of:
Step 1: performing a transcriptomic analysis on HEK293 cells transfected with an rAAV, a lentivirus, an adenovirus or a retrovirus and selecting the HEK293 cell if the transcription of at least one of the following genes is enriched compared to a negative control:
(i) TNFA_signaling_via_NFKB;
(ii) Interferon_Gamma_Response;
(iii) Interferon_Alpha_Response;
(iv) TGF_beta_Signaling; and/or
(v) IL6_JAK_STAT3_Signaling,
preferably if the transcription of
(1) interferon_gamma response; and/or
(2) interferon_alpha_respone
is enriched;
Step 2: culturing the rAAV, the lentivirus, the adenovirus or the retrovirus in the selected HEK293 cell in a culture medium comprising an amount of an interferon (IFN) inhibitor over an incubation period; and
Step 3: recovering the rAAV, the lentivirus, the adenovirus or the retrovirus from the cell culture.

In a third aspect, the present invention provides the use of an IFN-inhibitor transfected or infected with the rAAV, the lentivirus, the adenovirus or the retrovirus.

In a fourth aspect, the present invention provides a method for preparing a pharmaceutical composition comprising (i) performing the method according to the present invention, wherein the rAAV, the adenovirus, the lentivirus or the retrovirus comprises a therapeutic gene; and (ii) adding one or more pharmaceutically acceptable excipient(s) to the prepared rAAV particles, the adenovirus particles, lentivirus particles or retrovirus particles to thereby obtain a pharmaceutical composition.

In a fifth aspect, the present invention provides a kit for use in cell culture comprising
(a) HEK293 cells; and
(b) an IFN inhibitor, preferably selected from the group consisting of Ruxolitinib, Tofacitinib, Baricitinib, Delgocitinib, Emapalumab and Fontolizumab, and any structural analog thereof having IFN inhibitor activity. More preferably, the inhibitor is Ruxolitinib and any structural analog thereof having inhibitory activity for the JAK family kinases in the IFN signaling pathway. Particularly preferred, the structural analog has at least 50%, more preferably 80%, even more preferred at least 90%, most preferred at least 95% of the inhibitory activity for the Jak1 component compared to Ruxolitinib.

By conducting a transcriptomic analysis the present inventors have surprisingly found that the various available HEK293 cells are characterized by unique patterns of activated genes upon triple transfection for rAAV production. For example, the present inventors have found that in some of the HEK293 cells which exhibit a relatively low yield of rAAV production in fermentative production, interferon response genes are activated.

This was unexpected in light of Strasser et al., Int. J. Mol. Sci 22 (2021), 11499. In the latter article the effect of rAAV5 production on the proteome of HEK293 cells was investigated. The authors found that EIF2 signaling pathway, oxidative phosphorylation and the spliceosomal cycle were activated. A further article Barnes et al., Mol. Ther. Nucl. Acids 26 (2021), 94 used genome-wide activation screens to identify gRNAs potentially useful in increasing rAAV production in HEK293T cells. It was found that SKA2 or ITPRIP may be useful in increasing the rAAV production in HEK293T cells.

The finding of the present inventors therefore is surprising in view of this detailed research which did not provide any relevant pointer. The present inventors further exploited this unexpected finding by the administration of IFN inhibitors, in particular IFN signaling inhibitors which resulted in an increase of rAAV production in those HEK293 cells. By the addition of the IFN inhibitor, the rAAV virus production was increased after several days by more than 100% and even up to 200% which is certainly significant for a desired large-scale rAAV production in HEK293 cells.

### BRIEF DESCRIPTION OF THE DRAWINGS

FIG. 1: The yield of rAAV production in HEK293 cells (CL1) for non-treated (ctrl) cells, inhibitor solvent treated cells (DMSO) and in different inhibitor concentration treated cells, as determined by vector genome droplet digital PCR (ddPCR) in the supernatant and cell culture respectively is shown in FIGS. 1A and 1B, respectively. The yield of rAAV production in HEK293 cells (CL1) as determined by capsid particle in the supernatant and cell culture is shown in FIGS. 1C and 1D, respectively.

FIG. 2: In a separate set of experiments the rAAV yield of three different cell lines (CL1, CL2 and CL3) in the presence or absence (ctrl) was compared. The rAAV yield of CL1 ctrl was used as basis (100%) for comparing the yields among CL1, CL2 and CL3, see FIGS. 2(A), 2(B) and 2(C). Potential effects of respective solvent without inhibitor (DMSO, water for injection (wfi), PBS) were also tested. The results determined by AAV8 ELISA assay in the supernatants is shown in FIG. 2. FIG. 2(A) relates to the CL1 cell line; FIG. 2(B) to the CL2 cell line and FIG. 2(C) to the CL3 cell line.

FIG. 3: To test the effect of IFN inhibitor on the rAAV yield in large-scale bioreactor fermentation of HEK293 cells, 10 L bioreactors have been used. The results are shown in FIG. 3A (determination by capsid particlerAAV8 ELISA) and FIG. 3B (determination by ddPCR).

FIG. 4: To test effect of IFN inhibitor on the rAAV yield in adherent HEK293 cells the cells were cultivated in a T75 flask system. The results are shown in FIGS. 4A (determination by ddPCR) and B (determination by capsid particle AAV8 ELISA)

FIG. 5: To test the effect of IFN inhibitor on HEK293 suspension cells (CL1) for lentivirus production a shake flask system was used. The results are shown in FIG. 5 (determination by p24 ELISA)

### DETAILED DESCRIPTION OF THE INVENTION

In a first aspect the present invention provides a method for the fermentative production of a recombinant adeno-associated virus (rAAV), an adenovirus, a lentivirus or a retrovirus comprising the steps of:
Step 1: culturing the rAAV, the adenovirus, the lentivirus or the retrovirus in a HEK293 cell in a culture medium comprising an amount of an interferon (IFN) inhibitor over an incubation period; and
Step 2: recovering the rAAV, the adenovirus, the lentivirus or the retrovirus from the cell culture.

“AAV” herein includes an AAV serotype, a chimera or hybrid thereof. The AAV serotype may be selected from AAV1, AAV2, AAV3, AAV4, AAV5, AAV6, AAV7, AAV8, AAV9, AAV10, AAV11, AAV12, AAV13, and chimeric or hybrid variants thereof. Preferably, the AAV serotype is selected from AAV5, AAV6, AAV8, and AAV9, AAV-DJ, and more preferably AAV8. A chimeric AAV variant comprises more than one AAV serotype. A hybrid AAV variant comprises a mutant AAV serotype with modifications.

AAV are small viruses that infect humans and some other primate species. They belong to the genus Dependoparvovirus, which in turn belongs to the family Parvoviridae. They are small (20-25 nm) replication-defective, nonenveloped viruses and have linear single-stranded DNA (ssDNA) genome of approximately 4.8 kilobases (kb). rAAV is thought to primarily remain episomal.

For safety reasons, rAAV are usually prepared from 2, 3 or even several separate expressible units. Thus, many vector systems are manufactured by transient co-transfection of multiple plasmids. Stable transfection is possible as well.

For AAV, the so-called triple transfection method is well-known. To this end, the host cell is transfected with (i) a plasmid carrying the therapeutic gene of interest, (ii) a plasmid carrying AAV genes such as rep and cap and (iii) a plasmid carrying adenovirus 5 genes such as E4, E2a and VA.

Alternatively, recombinant AAV may be obtained by a double transfection method. In this method the host cell is transfected with (i) a plasmid carrying the therapeutic gene of interest and (ii) a plasmid carrying the adenovirus 5 genes such as E4, E2a and VA and further comprising the rep and cap genes of AAV.

“adenovirus” herein include adenoviral packaging systems, such as Adenovirus serotype 5 (Adv5), use adenoviral shuttle vectors for transporting the target gene sequence into E1 expressing production cell lines. Early viral transcription units, E1 and E3 are defected in the most commonly used recombinant adenoviral packaging systems. The system can be a two-vector system, consisting of adenovirus expression plasmids with different promotors and tags, and an adenovirus genome backbone with a E1 region deletion on the plasmid.

“lentivirus” herein includes HIV and variants thereof, and including lentivirus with pseudo-typed VSV-G envelope protein. Preferably, the lentivirus is derived from HIV-1.

Lentivirus is a genus of retroviruses that cause chronic and deadly diseases characterized by long incubation periods, in humans and other mammalian species. The genus includes the human immunodeficiency virus (HIV), which causes AIDS. Lentiviruses are distributed worldwide, and are known to be hosted in apes, cows, goats, horses, cats, and sheep as well as several other mammals. To increase safety of lentivirus, the components necessary for virus production are split across multiple plasmids (3 for 2nd-generation systems, 4 for 3rd-generation systems). The components of both systems are as follows:

Lentiviral transfer plasmid encoding the insert of interest. The transgene sequence is flanked by long terminal repeat (LTR) sequences, which facilitate integration of the transfer plasmid sequences into the host genome. Typically, it is the sequences between and including the LTRs that is integrated into the host genome upon viral transduction. Many lentiviral transfer plasmids are based on the HIV-1 virus. For safety reasons, transfer plasmids are all replication incompetent and may contain an additional deletion in the 3′LTR, rendering the virus “self-inactivating” (SIN) after integration.

Packaging plasmid(s). The packaging plasmid can contain gag, pol, rev and tat genes on one plasmid or distributed on two plasmids.

Envelope plasmid. Preferably, the envelope plasmid encodes VSV-G.

Lentiviruses can integrate a significant amount of viral nucleic acid DNA into the DNA of the host cell and can efficiently infect nondividing cells, so they are one of the most efficient methods of gene delivery. They can become endogenous, integrating their genome into the host germline genome, so that the virus is henceforth inherited by the host's descendants.

“retrovirus” herein includes gamma retroviruses. Preferably, the retrovirus is Murine Leukemia virus. In a further preferred embodiment the retrovirus is derived from HIV-1.

“A HEK293 cell” herein includes Human Embryonic Kidney cells originally isolated and grown by Alex van der Eb. These were transfected with sheared adenovirus 5 (Ad5) DNA by Frank Graham (Graham F L, Smiley J, Russell W C, Nairn R (July 1977). The Journal of General Virology. 36 (1): 59-74). Incorporating the adenoviral genes into the HEK293 cell genome resulted in the cells becoming efficient at producing high amounts of recombinant proteins from plasmid vectors. The term also includes any derivative of HEK293 cells such as e.g. HEK293-F, HEK293-H, HEK293-T and HEK293FT, and HEK293-E, also known as HEK293-EBNA1. The HEK293 cell line and derivatives thereof are obtainable from cell culture collections such as ATCC or ECACC and commercial providers.

Preferably, the HEK293 cells when used in the method of the present invention produce an increased yield of the rAAV, the adenovirus, the lentivirus or the retrovirus in the presence of the IFN inhibitor in the culture medium. An “increased yield” may be defined as the yield of a rAAV, lentivirus or retrovirus as produced in the HEK293 cell line in the presence of the IFN inhibitor which is higher as compared to the yield as produced in the HEK293 cell line in the absence of the IFN inhibitor over the same incubation period under the same culturing conditions. Preferably an “increased yield” means that the yield is at least 0.2 times higher, at least 0.3 times higher, at least 0.5 times higher, at least 0.7 times higher, at least 1.0 times higher, at least 1.5 times higher, at least 2.0 times higher, at least 2.5 times higher, at least 3.0 times higher, at least 4 times higher, or at least 5 times higher as compared to the yield as produced in the HEK293 cell line in the absence of the IFN inhibitor. Preferably, the yield is determined in the presence of 5 μM Ruxolitinib as IFN inhibitor over an incubation period of 96 hours as outlined in the examples.

In certain embodiments, the “increased yield” may be defined by an rAAV yield from the HEK293 cells in the presence of the inhibitor of at least about 1×10⁹ vector genome-containing particles per milliliter of cell culture (vg/mL), e.g., at least about 5×10⁹, 1×10¹⁰, 5×10¹⁰, 1×10¹¹, 1×10¹², 1×10¹³ or 1×10¹⁴ or more vector genome-containing particles per milliliter of cell culture at all upstream and downstream process steps as well as after the concentration of the product. Preferably, the yield is determined as described in the examples.

The yield can also be measured as capsid particle per L or mL of cell culture. In certain embodiments, an AAV yield from the HEK293 cells in the presence of the inhibitor of at least about 1×10⁹ vector genome-containing particles per milliliter of cell culture (vg/mL), e.g. at least about 5×10⁹, 1×10¹⁰, 5×10¹⁰, 1×10¹¹, 1×10¹², 1×10¹³ or 1×10¹⁴ or more capsid particles per milliliter of cell culture is obtained.

In a preferred embodiment the HEK293 cell of the present invention is characterized by an activation of at least one of the following cellular pathways at the indicated time points:
(1) interferon_gamma response (0-48 h);
(2) interferon_alpha_respone (0-48 h);

Activation of the cellular pathways is achieved by triple transfection of the plasmids necessary for rAAV8 replication in the transfected HEK293 cells. Further details are outlined in the examples section.

In another preferred embodiment, the HEK293 cell is characterized of the present invention by an activation of at least one of the following cellular pathways:
(i) TNFA_signaling_via_NFKB;
(ii) Interferon_Gamma_Response;
(iii) Interferon_Alpha_Response;
(iv) TGF_beta_Signaling;
(v) IL6_JAK_STAT3_Signaling;

Preferably, the HEK293 cells show an activation of at least 2, at least three, at least four, more preferably at least five of the above listed cellular pathways.

More preferably, the at least 2 activated cellular pathways are selected from the group consisting of TNFA_signaling_via_NFKB, interferon_gamma response, interferon_alpha_response, TGF_beta_Signaling, IL6_JAK_STAT3_Signaling,

Particularly preferred, the at least 2 activated genes are selected from the group consisting of interferon_gamma response, interferon_alpha_response and IL6_JAK_STAT3_Signaling.

Most preferably, the HEK293 cell to be used in the method of the present invention is CRL-1573.

In one preferred embodiment, the HEK293 cell is an adherent cell line. In an even more preferred embodiment, the HEK293 cell is a suspension cell line, or adapted to grow in suspension. Depending on the fact whether the HEK293 cell is an adherent cell line or a suspension cell line different culturing methods are available to the skilled person. The culturing may be carried out in a petri dish, a shaker flask or a roller bottle. The culturing in a shaker flask or a roller bottle are preferred.

The “culture medium” herein includes any medium suitable for culturing mammalian cells. The medium can be a complex, semi-defined or defined medium. Preferably, the culture medium is a chemically defined medium. More preferably, commercially available media are used such as the mammalian cell media as known to the person skilled in the art.

The culture conditions can be varied depending upon the special requirements of the cells in parameters such as pH, salt concentration, temperature, etc. The process may be carried out as a continuous process or in batch mode or fed-batch mode which are well-known to the skilled person. Preferably, the process is carried out in batch mode or fed-batch mode.

“IFN inhibitor” herein includes an inhibitor of the interferon immune response pathway. Interferons are a group of signaling proteins made by host cells in response to the presence of several viruses. In a typical scenario a virus-infected cell will release interferons causing nearby cells to heighten their anti-viral defenses. Type I interferons can be broadly characterized into three groups: IFN-I, Type II (IFN-II), and Type III (IFN-III) with subcategories therein based on gene loci of the IFN transcribing genes as well as difference in their cognate receptors. IFN-I is the largest and most well-characterized group with seven classes: IFNα, IFNβ, IFNδ, IFNϵ, IFNκ, IFNω, and IFNτ whereas IFN-II comprises IFNγ. IFN-I and IFN-II signal through IFNαR1/R2 (IFNAR) and IFNγR1/R2 (IFNGR), respectively. The last class IFN-III, otherwise classified as “IFN-like cytokines”, consists of interleukin (IL)-28A (IFNλ2), IL28B (IFNλ3), and TL29 (IFNλ1) and signals through IL-28RI/IL10R2 receptor chains.

Recognition of microbial products by a range of cell-surface and intracellular pattern recognition receptors, including Toll-like receptors (TLRs) and retinoic acid-inducible gene I (RIG-I), can lead to induction of the genes encoding type I interferons (IFNs), which is mediated by several distinct signalling pathways. On the binding of type I IFNs to their receptor (IFNAR), multiple downstream signalling pathways can be induced, leading to a diverse range of biological effects. The canonical signal transducer and activator of transcription 1 (STAT1)-STAT2-IFN-regulatory factor 9 (IRF9) signalling complex (also known as the IFN-stimulated gene factor 3 (ISGF3) complex) binds to IFN-stimulated response elements (ISREs) in gene promoters, leading to induction of a large number of IFN-stimulated genes (ISGs). Type I IFNs can also signal through STAT1 homodimers, which are more commonly associated with the IFNγ-mediated signalling pathway. Other STAT heterodimers and homodimers may also be activated downstream, including STAT3, STAT4 and STAT5. Other signalling pathways that do not rely on Janus kinase (JAK) and/or STAT activity may also be activated, including mitogen-activated protein kinases (MAPKs) and the phosphoinositide 3-kinase (PI3K) pathway, thereby leading to diverse effects on the cell.

The IFN inhibitors for use in the present invention are intended to be used to target the IFN-induction and/or signalling cascades. The activation of multiple signalling pathways by the engagement of IFN with its receptors is critical for the generation of IFN-mediated biological function. As used herein, the phrase “IFN induction cascade” encompasses any virus-induced pathway that leads to the activation of IFN-α/β transcription and expression. The molecules in the IFN induction cascade that may be targeted by the inhibitors may include, but are not limited to TLR3, MDA5, RIG-I, Cardif, TBK1/IKKE, 25 IKKa/, IRF3, NFKB or ATF-2/C-JUN. As used herein, the phrase “IFN signalling cascade” encompasses any IFN-activated signalling pathway whose activation leads to antiviral changes in the cell. The signalling pathway targeted may include, but is not limited to, the JAK-STAT pathway.

The inhibitors for use in the present invention may target one or more components of the IFN induction and/or signalling cascades shown in Table 1. The inhibitors used in the present invention may be used in isolation or in combination.

TABLE 1
Possible targets for Interferon pathway inhibition

| | | | | |
|---|---|---|---|---|
| ADAR | IFI6 | IFNG | NRIP3 | TLR8 |
| ARIH1 | IFI16 | IFNGR1 | NUP107 | TLR9 |
| CHUK | IFIH1 | IFNGR2 | NUP133 | TPR |
| CREBBP | IFIT1 | IRAK1 | OAS3 | TRAF3 |
| DHX58 | IFIT1 | IRAK4 | PDE12 | TRAF6 |
| EGR1 | IFIT2 | IRF1 | PIN1 | TRIM14 |
| EIF2AK2 | IFIT3 | IRF2 | PLCG1 | TRIM17 |
| EIF4A2 | IFIT5 | IRF3 | PTPN11 | TRIM2 |
| EIF4A3 | IFITM1 | IRF4 | PTPN6 | TRIM21 |
| EIF4E | IFITM2 | IRF5 | RAE1 | TRIM22 |
| EIF4E2 | IFITM3 | IRF6 | SAMHD1 | TRIM25 |
| EIF4E3 | IFNA1 | IRF7 | SEH1L | TRIM26 |
| EIF4G1 | IFNA10 | IRF8 | SMAD3 | TRIM29 |
| EIF4G2 | IFNA14 | IRF9 | SOCS1 | TRIM3 |
| EIF4G3 | IFNA16 | IRF2BPL | SOCS3 | TRIM34 |
| ESAMHD1 | IFNA17 | ISG15 | STAT1 | TRIM35 |
| FADD | IFNA2 | JAK1 | STAT2 | TRIM38 |
| FLNA | IFNA21 | JAK2 | STAT3 | TRIM45 |
| HERC5 | IFNA4 | JAK3 | STAT4 | TRIM48 |
| HLA-A | IFNA5 | KPNA2 | STAT6 | TRIM5 |
| HLA-B | IFNA6 | KPNB1 | TAB2 | TRIM6 |
| HLA-C | IFNA7 | MAPK3 | TAB3 | TRIM62 |
| HLA-F | IFNA8 | MAVS | TANK | TRIM68 |
| HLA-H | IFNAR1 | NAP1L1 | TLR3 | TRIM8 |
| IFI30 | IFNAR2 | NFKBIA | TLR4 | TYK2 |
| IFI35 | IFNB1 | NRIP1 | TLR7 | UBE2E1 |

In a preferred embodiment of the invention, the inhibitors for use in the present invention target the JAK/STAT signalling pathway. The inhibitors may target any component of the JAK/STAT pathway. The targeted components of the JAK/STAT pathway may include, but are not limited to, Tyk2, Jak1, Jak2, STAT-1, STAT-2 and IRF-9. In a further preferred embodiment, the inhibitor targets Jak1. In a further preferred embodiment of the invention, the inhibitors for use in the present invention target the TBK-1/IKKE/IKK2 induction pathway.

The inhibitors for use in the present invention can be any molecule which decreases the activity of the IFN induction and/or signalling cascades. Non-limiting examples of inhibitors which could be used in accordance with the present invention include small molecule inhibitors, siRNAs, miRNAs, lipocalins, plastic antibodies, antibodies or antibody fragments.

The IFN inhibitor is preferably not selected from niacin and/or niacinamide.

Preferably, the IFN inhibitor is not selected from (i) methyl nicotinate, (ii) myo-inositol and (iii) choline.

In a further preferred embodiment the IFN inhibitor is not selected from the (iv) combination of methyl nicotinate and myo-inositol and (v) the combination of methyl nicotinate and choline and (vi) the combination of myo-inositol and choline.

In a further preferred embodiment the IFN inhibitor is not (vii) the combination of methyl nicotinate, myo-inositol and choline.

If the inhibitor was designed to target Jak1, one could envisage an antibody, plastic antibody or antibody fragment raised against Jak1 such that in use, the antibody, plastic antibody or antibody fragment binds to Jak1 and prevents it phosphorylating STAT transcription factors, for example.

Alternatively, if the inhibitor were a miRNA or siRNA or shRNA, one could envisage the miRNA or siRNA or shRNA having a sequence complementary to a portion of the Jak1 mRNA sequence, such that the miRNA or siRNA or shRNA inhibitor would bind Jak1 mRNA, thereby reducing translation and reducing the abundance of Jak1 in the cell.

Preferably the inhibitor is a small molecule. As used herein the phrase “small molecule” refers to a low molecular weight (less than approximately 800 Da) organic or inorganic compound.

The small molecule inhibitor for use in the present invention can function through competitive, uncompetitive, mixed or non-competitive inhibition. In one embodiment, the inhibitor is a competitive inhibitor.

Preferably, the IFN inhibitor is selected from the group consisting of Ruxolitinib (CAS: 941678-49-5), Tofacitinib, Baricitinib, Delgocitinib, Emapalumab and Fontolizumab, preferably the inhibitor is Ruxolitinib.

Ruxolitinib is an inhibitor of the JAK family kinases component in the IFN signaling pathway. Ruxolitinib is also known as INC 424, INCB 018424, and (3R)-3-cyclopentyl-3-[4-(7H-pyrrolo[2,3-d]pyrimidin-4-yl)pyrazol-1-yl]propanenitrile.

Tofacitinib is an inhibitor of the Jak1 component in the IFN signaling pathway.

Baricitinib is an inhibitor of Jak, blocking the subtypes Jak1 and Jak2.

Delgocitinib is an inhibitor of Jak.

Emapalumab is a human monoclonal antibody directed against interferon-γ (IFN-γ).

Fontolizumab is a humanized antibody directed against interferon-γ (IFN-γ).

In a preferred embodiment the final concentration of the IFN inhibitor in the medium is in the range from about 0.0001 to 50 mM, preferably about 1 to about 100 μM, more preferably about 0.5 to about 25 μM. Particularly preferred, the IFN inhibitor concentration is 1 μM, 5 μM, 10 μM, 15 μM, 20 μM, or 25 μM, with 5 μM and 10 μM more preferred. In particular, 5 μM or 10 μM Ruxolitinib is preferred.

The interferon inhibitor may either (i) be added to the culture medium containing the HEK293 cells before the plasmids for rAAV production are added in the transfection step, or (ii) added to the culture medium containing the HEK293 cells and the plasmids for rAAV production during the transfection step. Process variant (i) is preferred.

A preferred process for the rAAV production according to the invention comprises the following steps:
(a) culturing HEK293 cells in a culture medium containing an amount of an IFN inhibitor over a first incubation period;
(b1) transfecting the HEK293 cells with three plasmids necessary for rAAV production, preferably with (i) an adenovirus helper plasmid, (ii) a packaging rep-cap plasmid and (iii) a recombinant rAAV plasmid comprising a transgene; or
(b2) transfecting the HEK293 cells with two plasmids necessary for rAAV production, preferably with (i) a plasmid comprising the adenovirus helper genes and the rep-cap genes and (ii) a recombinant rAAV plasmid comprising a transgene;
(c) culturing of the transfected HEK293 cells over a second incubation period; and
(d) recovering the rAAV particles.

A further preferred process for the rAAV production according to the invention comprises the following steps:
(a) culturing HEK293 cells in a culture medium over a first incubation period;
(b1) transfecting the HEK293 cells in a culture medium containing an amount of an IFN inhibitor and the transfection reagent with (i) an adenovirus helper plasmid, (ii) a packaging rep-cap plasmid and (iii) a recombinant rAAV plasmid comprising a transgene; or
(b2) transfecting the HEK293 cells with two plasmids necessary for rAAV production in a culture medium containing an amount of an IFN inhibitor and the transfection reagent, preferably with (i) a plasmid comprising the adenovirus helper genes and the rep-cap genes and (ii) a recombinant rAAV plasmid comprising a transgene;
(c) culturing of the transfected HEK293 cells over a second incubation period in the presence of the IFN inhibitor; and
(d) recovering the rAAV particles.

A further preferred process for the rAAV production according to the invention comprises the following steps:
(a) culturing HEK293 cells in a culture medium over a first incubation period;
(b1) triple transfecting the HEK293 cells in a culture medium containing the transfection reagent with (i) an adenovirus helper plasmid, (ii) a packaging rep-cap plasmid and (iii) a recombinant rAAV plasmid comprising a transgene; or
(b2) transfecting the HEK293 cells with two plasmids necessary for rAAV production in a culture medium containing the transfection reagent, preferably with (i) a plasmid comprising the adenovirus helper genes and the rep-cap genes and (ii) a recombinant rAAV plasmid comprising a transgene;
(c) culturing of the transfected HEK293 cells over a second incubation period in a culture medium comprising an IFN inhibitor; and
(d) recovering the rAAV particles.

### Step (a)

The HEK293 cells may be cultured with the IFN inhibitor in step (a) over a period of about 1 hour to about 48 hours, preferably 1 to 12 hours preferably 1 to 6 hours, more preferably 1, 2 or 3 hours before step (b) is performed.

### Step (b)

The (i) adenovirus helper plasmid may comprise one or more of the adenovirus genes selected from the group consisting of E1A, E1B, E2A, E40RF6 and VA. More preferably, the adenovirus helper plasmid comprises the adenovirus genes E2A, E40RF6 and VA.

The (ii) packaging rep-cap plasmid comprises the rep and cap genes of rAAV. Alternatively, the necessary rep and cap genes are provided by a HEK293 cell stably transfected with said rep and cap genes.

Preferably, the rep-cap plasmid comprises a rep2 gene and a cap8 gene.

The (iii) rAAV plasmid may contain any heterologous gene(s) of interest. Genes of interest include nucleic acids encoding polypeptides or RNAs, including reporter, therapeutic (e.g., for medical or veterinary uses), immunogenic (e.g., for vaccines), or diagnostic polypeptides or RNAs. As a further alternative, the heterologous nucleic acid can encode any polypeptide or RNA that is desirably produced in a cell in vitro, ex vivo, or in vivo.

In an embodiment of the invention, the rAAV vector is self-complementary. Self-complementary vectors may, advantageously, overcome the rate-limiting step of second-strand DNA synthesis and confer earlier onset and stronger gene expression. Preferably, the rAAV vector comprising a gene of interest is self-complementary. In an embodiment, the vector comprises single-stranded DNA.

The rAAV vector may further comprise regulatory sequences which are operably linked to the gene of interest in a manner which permits one or more of the transcription, translation, and expression in a cell infected with a virus that comprises the vector.

The regulatory sequences may include appropriate transcription initiation, termination, promoter and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability; and when desired, sequences that enhance secretion of the encoded product. PolyA signal sequences may be synthetic or may be derived from many suitable species, including, for example, SV-40, human and bovine. The regulatory sequences may also include an intron. The regulatory sequences may also include a promoter. The promoter may be any promoter suitable for expressing the gene of interest in a target cell. The promoter may be inducible or constitutive.

In an embodiment of the invention, the rAAV vector comprises an ITR or a functional fragment thereof. Preferably, the vector comprises a 5′ AAV ITR and a 3′ AAV ITR. The ITRs may be of any suitable rAAV serotype, including any of the AAV serotypes described herein. The ITRs may be readily isolated using techniques known in the art and may be isolated or obtained from public or commercial sources (e.g., the American Type Culture Collection, Manassas, VA). Alternatively, the ITR sequences may be obtained through synthetic or other suitable means by reference to published sequences. Preferably, the vector comprises a 5′ AAV2 ITR and a 3′ AAV2 ITR.

Preferably, the recombinant rAAV plasmid comprises a 5′ AAV ITR, a gene of interest and a 3′ AAV ITR.

The 5′ AAV ITR and the 3′ AAV ITR may be from the same serotype or from different serotypes. More preferably, the 5′ AAV ITR and the 3′ AAV ITR are from the AAV8 serotype.

The transfection step may be carried out using polyethyleneimine or FectoVIR®-AAV as transfection reagent. Preferably, polyethylenimine is used as transfection reagent.

In one embodiment the transfection reagent is added, and the IFN-inhibitor remains in the medium. The transfection steps may be carried out using the transfection reagent, and the IFN-inhibitor. Further, the transfection steps may be carried out using the transfection reagent, and the IFN-inhibitor in transfection split media.

### Step (c)

The second incubation period is at least 24 hours, preferably at least 48 hours, more preferably at least 72 hours, more preferably at least 96 hours, and most preferred at least 120 hours.

Overall the incubation period in the presence of the IFN inhibitor, independently on when it is added to the medium, is at least 24 hours, preferably at least 48 hours, more preferably at least 72 hours, even more preferred at least 96 hours, and most preferred at least 120 hours.

The rAAV template and rAAV rep and cap sequences are provided under conditions such that virus vector comprising the rAAV template packaged within the rAAV capsid is produced in the cell. The method can further comprise the step of collecting the virus vector from the culture. In one embodiment, the virus vector can be collected by lysing the cells, e.g., after removing the cells from the culture medium, e.g., by pelleting the cells. In another embodiment, the virus vector can be collected from the medium in which the cells are cultured, e.g., to isolate vectors that are secreted from the cells. Some or all of the medium can be removed from the culture one time or more than one time, e.g., at regular intervals during the culturing step for collection of rAAV (such as every 12, 18, 24, or 36 hours, or longer extended time that is compatible with cell viability and vector production), e.g. beginning about 48 hours post-transfection. After removal of the medium, fresh medium, with or without additional nutrient supplements, can be added to the culture. In one embodiment, the cells can be cultured in a perfusion system such that medium constantly flows over the cells and is collected for isolation of secreted rAAV.

### Step (d)

Insofar as the rAAV is secreted from the HEK293 cells step (d) includes collecting of the supernatant of the culture medium and purifying the rAAV particles from the supernatant. Insofar as the rAAV is not secreted from the HEK293 cells step (d) includes a step of disrupting the HEK293 cells to obtain the rAAV particles and subsequent purifying of the rAAV particles.

Purification may comprise, but not limited to any of the following methods: ion exchange, hydrophobic chromatography, affinity chromatography, filtration, precipitation, density gradient centrifugation, and heparin sulfate matrix.

In representative embodiments, the method of the invention is completely scalable, so it can be carried out in any desired volume of culture medium, e.g., from 10 ml (e.g., in shaker flasks) to 10 L, 50 L, 100 L, or more (e.g., in bioreactors such as wave bioreactor systems and stirred tanks). The method is suitable for production of all serotypes and chimeras of AAV, e.g., AAV1, AAV2, AAV3, AAV4, AAV5, AAV6, AAV7, AAV8, AAV9, AAV10, AAV11, AAV12, AAV13, AAV-DJ and any chimeras thereof.

The yield of the rAAV from the HEK293 cells may be determined as vector genome/L cell culture in e.g. a droplet digital PCR assay (available e.g. from Bio-Rad). Alternatively, the yield may be determined by assessing the amount of capsid particle per L or mL of cell culture in e.g. an ELISA assay. Details of the assays are outlined in the examples section.

In certain embodiments, the method provides at least about 1×10⁹ purified vector genome-containing particles per milliliter of cell culture (vg/mL), e.g., at least about 5×10⁹, 1×10¹⁰, 5×10¹⁰, 1×10¹¹, 1×10¹², 1×10¹³ or 1×10¹⁴ or more vector genome-containing particles per milliliter of cell culture. The yield can also be measured as capsid particle per L or mL of cell culture. In certain embodiments, the method provides at least about 1×10⁹ purified vector genome-containing particles per milliliter of cell culture (vg/mL), e.g. at least about 5×10⁹, 1×10¹⁰, 5×10¹⁰ 1×10¹¹, 1×10¹², 1×10¹³ or 1×10¹⁴ or more capsid particles per milliliter of cell culture.

The yield may be determined in the supernatant or cell suspension. The cell suspension comprises the virus content in the supernatant as well as the virus content in the disrupted cells. Preferably, the yield is determined in the cell suspension after at least one freeze thaw cycle. The absolute titer depends e.g. on the analytical methods used, the AAV serotype, the inserted therapeutic gene of interest and the transfection method.

In a further aspect the present invention provides the use of an IFN inhibitor for increasing the yield of rAAV, adenovirus, lentivirus or retrovirus in a culture of HEK293 cells. The IFN inhibitor and the HEK293 cells are as defined above. Preferably, the IFN inhibitor is selected from the group consisting of Ruxolitinib, Tofacitinib, Baricitinib, Delgocitinib, Emapalumab and Fontolizumab and any structural analog thereof having IFN inhibitor activity, preferably the IFN inhibitor is Ruxolitinib and any structural analog thereof having inhibitory activity for the Jak1 component in the IFN signaling pathway.

In a further aspect the present invention provides a method for preparing a pharmaceutical composition comprising performing (i) the method according to the present invention, wherein the rAAV, the adenovirus, the lentivirus, or the retrovirus comprises a therapeutic gene; and (ii) adding one or more pharmaceutically acceptable excipients to the prepared rAAV particles, adenovirus particles, lentivirus particles or retrovirus particles to thereby obtain a pharmaceutical composition.

Any pharmaceutically acceptable excipient can be used within the context of the invention, and such pharmaceutically acceptable excipients are well known in the art. The choice of the pharmaceutically acceptable excipients will be determined, in part, by the particular site to which the composition is to be administered and the particular method used to administer the composition. The pharmaceutical composition can optionally be sterile or sterile with the exception of the one or more recombinant adeno-associated viral vectors.

Suitable formulations for the pharmaceutical composition include aqueous and non-aqueous solutions, isotonic sterile solutions, which can contain antioxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the pharmaceutical composition for use in the inventive method is formulated to protect the adeno-associated viral vectors from damage prior to administration. For example, the pharmaceutical composition can be formulated to reduce loss of the adeno-associated viral vectors on devices used to prepare, store, or administer the expression vector, such as glassware, syringes, or needles. The pharmaceutical composition can be formulated to decrease the light sensitivity and/or temperature sensitivity of the adeno-associated viral vectors. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, mannitol and combinations thereof. Use of such a composition may extend the shelf life of the vector, facilitate administration, and increase the efficiency of the inventive method. A pharmaceutical composition also can be formulated to enhance transduction efficiency of the recombinant adeno-associated viral vector. In addition, one of ordinary skill in the art will appreciate that the pharmaceutical composition can comprise other therapeutic or biologically-active agents. For example, factors that control inflammation, such as ibuprofen or steroids, can be part of the pharmaceutical composition to reduce swelling and inflammation associated with in vivo administration of the adeno-associated viral vectors. Antibiotics, i.e., microbicides and fungicides, can be present to treat existing infection and/or reduce the risk of future infection, such as infection associated with gene transfer procedures.

In a further aspect the present invention provides a kit for use in cell culture comprising
(a) HEK293 cells; and
(b) an IFN inhibitor selected from the group consisting of Ruxolitinib, Tofacitinib, Baricitinib, Delgocitinib, Emapalumab and Fontolizumab and any structural analog thereof having IFN inhibitor activity, preferably the IFN inhibitor is Ruxolitinib and any structural analog thereof has at least 50%, more preferably 80%, even more preferred at least 90%, most preferred at least 95% of the inhibitory activity for the JAK family kinases compared to Ruxolitinib.

The kit may include further components. It may also include instructions for the user.

### Items of the Invention

1. A method for the fermentative production of an adeno-associated virus (AAV), an adenovirus, a lentivirus or a retrovirus comprising the steps of:
Step 1: culturing the rAAV, the lentivirus, the adenovirus or the retrovirus in a HEK293 cell in a culture medium comprising an amount of an interferon (IFN) inhibitor over an incubation period; and
Step 2: recovering the rAAV, the adenovirus, the lentivirus or the retrovirus from the cell culture.

2. The method of item 1, wherein the yield of the rAAV, the adenovirus, the lentivirus or the retrovirus over an incubation period of at least 96 hours is increased by at least 10%, preferably by at least 50% compared to the yield of the rAAV, the adenovirus, the lentivirus or the retrovirus in the absence of the IFN inhibitor, more preferably by at least 100%, even more preferred by at least 200%.

3. The method of item 1 or 2, wherein in the presence of the IFN inhibitor from the culture medium the HEK293 cell produces an rAAV, a adenovirus, a lentivirus or a retrovirus yield of at least about 1×10⁹ purified vector genome-containing particles per milliliter of cell culture (vg/mL), preferably at least about 5×10⁹, more preferably at least 1×10¹⁰, more preferred of at least 5×10¹⁰, and most preferred at least 1×10¹¹, 1×10¹², 1×10¹³, 1×10¹⁴ or 5×10¹⁴ or more vector genome-containing particles per milliliter of cell culture.

4. The method of any one of items 1 to 3, wherein the HEK293 cell is characterized upon rAAV transfection by an activation of at least one of the following genes:
(1) interferon_gamma response; and/or
(2) interferon_alpha_response.

5. The method of item 4, wherein the HEK293 cell is characterized upon rAAV transfection by an activation of at least interferon_gamma response and interferon_alpha_response.

6. The method of any one of items 1 to 3, wherein the HEK293 cell line is characterized upon rAAV transfection by an activation of at least one of the following genes:
(i) TNFA_signaling_via_NFKB;
(ii) Interferon_Gamma_Response;
(iii) Interferon_Alpha_Response;
(iv) TGF_beta_Signaling; and/or
(v) IL6_JAK_STAT3_Signaling.

7. The method of item 6, wherein the at least 2 activated genes are selected from the group consisting of TNFA_signaling_via_NFKB, interferon_gamma response, interferon_alpha_response, TGF_beta_Signaling, and IL6_JAK_STAT3_Signaling, more preferably the at least 2 activated genes are selected from the group consisting of interferon_gamma response, interferon_alpha_response and IL6_JAK_STAT3_Signaling.

8. The method of item 6 or 7, wherein the HEK293 cell shows an activation of at least 2, at least three, at least four, more preferably each of the five genes are activated.

9. The method according to any one of items 1 to 8, wherein the HEK293 cell is in the presence of the IFN inhibitor from the culture medium characterized by an AAV yield of at least 1×10¹⁰, more preferred of at least 5×10¹⁰, and most preferred at least 1×10¹¹, 1×10¹², 1×10¹³, 1×10¹⁴ or 5×10¹⁴ capsid particles per milliliter culture.

10. The method according to any one of items 1 to 9, wherein the HEK293 cell is an adherent cell line.

11. The method according to any one of items 1 to 9, wherein the HEK293 cell is a suspension cell line, or adapted to grow in suspension.

12. The method according to any one of items 1 to 11, wherein the HEK293 cell is selected from the group consisting of CRL-1573 and NRC (HEK293SF-3F6).

13. The method according to any one of items 1 to 12, wherein Step 1 is carried out in a petri dish, a shaker flask or a roller bottle.

14. The method according to any one of items 1 to 12, wherein Step 1 is carried out in a bioreactor, preferably the bioreactor comprises a volume of at least 10 L.

15. The method according to any one of items 1 to 14, wherein the rAAV is selected from any AAV serotype selected from AAV1, AAV2, AAV3, AAV4, AAV5, AAV6, AAV7, AAV8, AAV9, AAV10, AAV11, AAV12 or AAV13, chimeric and hybrid variants thereof, preferably the AAV serotype is selected from AAV5, AAV6, AAV8, and AAV9, more preferably AAV8.

16. The method according to any one of items 1 to 14, wherein the lentivirus is derived from a human immunodeficiency virus (HIV), preferably the lentivirus is derived from HIV-1.

17. The method according to item 16, wherein the lentivirus vector is obtained by transfection of the HEK293 cell with at least one packaging plasmid, preferably in combination with an envelope plasmid derived from vesicular stomatitis virus (VSV), more preferably the envelope plasmid is derived from VSV-G.

18. The method according to any one of items 1 to 14, wherein the retrovirus is murine leukemia virus (MuLV).

19. The method according to any one of items 1 to 18, wherein the IFN inhibitor is a molecule which decreases the activity of the IFN induction and/or signalling cascades.

20. The method according to item 19, wherein the IFN inhibitor targets the JAK/STAT signaling pathway.

21. The method according to item 20, wherein the IFN inhibitor targets Jak1.

22. The method according to any one of items 19 to 21, wherein the IFN inhibitor is selected from the group consisting of Ruxolitinib, Tofacitinib, Baricitinib, Delgocitinib, Emapalumab and Fontolizumab, and any structural analog thereof having IFN inhibitor activity, preferably the IFN inhibitor is Ruxolitinib and any structural analog thereof having at least 50% of the inhibitory activity for the JAK family kinases in the IFN signaling pathway as compared to Ruxolitinib.

23. The method according to any one of items 1 to 22, wherein the final concentration of the IFN inhibitor in the medium is in the range from about 0.0001 to 50 mM, preferably about 1 to about 100 μM, more preferably about 0.5 to about 25 μM, even more preferred the final concentration is about 1 μM, about 5 μM, about 10 μM, about 15 μM, about 20 μM or about 25 μM.

24. The method according to any one of items 1 to 23, wherein the culture medium is a chemically defined medium.

25. The method according to any one of items 1 to 24 comprising the following steps:
(a) culturing HEK 293 cells in a culture medium containing an amount of an IFN inhibitor over a first incubation period;
(b1) transfecting the HEK293 cells with three plasmids necessary for rAAV production, preferably with (i) an adenovirus helper plasmid, (ii) a packaging rep-cap plasmid and (iii) a recombinant rAAV plasmid comprising a transgene; or
(b2) transfecting the HEK293 cells with two plasmids necessary for rAAV production, preferably with (i) a plasmid comprising the adenovirus helper genes and the rep-cap genes and (ii) a recombinant rAAV plasmid comprising a transgene;
(c) culturing of the transfected HEK293 cells over a second incubation period; and
(d) recovering the AAV particles.

26. The method of item 25, wherein the HEK293 cells are cultured with the IFN inhibitor in step (a) over a period of about 1 hour to about 12 hours, preferably 1 to 6, more preferably 1 to 3 hours before step (b) is performed.

27. The method of item 25 or 26, wherein the (i) adenovirus helper plasmid comprises one or more of the adenovirus genes selected from the group consisting of E1A, E1B, E2A, E40RF6 and VA; and/or (ii) the packaging rep-cap plasmid comprises a Rep2 gene and a Cap8 gene and/or (iii) the AAV plasmid comprises a 5′ AAV ITR and a 3′ AAV ITR from the same AAV serotype, preferably from AAV2.

28. The method of any one of items 25 to 27, wherein the transfection step is carried out using polyethyleneimine or FectoVIR™-AAV as transfection reagent.

29. The method of any one of items 25 to 28, wherein the second incubation period is at least 24 hours, preferably at least 48 hours, more preferably at least 72 hours, most preferred at least 96 hours.

30. The method of any one of items 25 to 29, wherein insofar the rAAV is secreted from the HEK293 cells step (d) includes collecting of the supernatant of the culture medium and purifying the rAAV particles from the supernatant.

31. The method of any one of items 25 to 30, wherein insofar the rAAV is not secreted from the HEK293 cells step (d) includes a step of disrupting the HEK293 cells to obtain the rAAV particles and subsequent purifying of the rAAV particles.

32. A method for the fermentative production of an adeno-associated virus (AAV), an adenovirus, a lentivirus or a retrovirus comprising the steps of:
Step 1: performing a gene set enrichment analysis on HEK293 cells transfected with an rAAV, an adenovirus, a lentivirus or a retrovirus and selecting the HEK293 cell if the transcription of at least one of the following genes is enriched compared to a negative control:
(i) TNFA_signaling_via_NFKB;
(ii) Interferon_Gamma_Response;
(iii) Interferon_Alpha_Response;
(iv) TGF_beta_Signaling; and/or
(v) IL6_JAK_STAT3_Signaling,
preferably if the transcription of
(1) interferon_gamma response; and/or
(2) interferon_alpha_respone
is enriched;
Step 2: culturing the rAAV, the adenovirus, the lentivirus or the retrovirus in the selected HEK293 cell in a culture medium comprising an amount of an interferon (IFN) inhibitor over an incubation period; and
Step 3: recovering the rAAV, the adenovirus, the lentivirus or the retrovirus from the cell culture.

33. Use of an IFN-inhibitor for increasing the yield of rAAV, adenovirus, lentivirus or retrovirus in a culture of HEK293 cells transfected or infected with the rAAV, the adenovirus, the lentivirus or the retrovirus.

34. Use of item 33, wherein the HEK293 cell is characterized upon transfection or infection with the rAAV, the adenovirus, the lentivirus or the retrovirus by an activation of at least one of the following cellular pathways:
(1) interferon_gamma response; and/or
(2) interferon_alpha_response.

35. Use according to item 34, wherein the IFN inhibitor is selected from the group consisting of Ruxolitinib, Tofacitinib, Baricitinib, Delgocitinib, Emapalumab and Fontolizumab, and any structural analog thereof having IFN inhibitor activity, preferably the IFN inhibitor is Ruxolitinib and any structural analog thereof having at least 50% of the inhibitory activity for the JAK family kinases in the IFN signaling pathway as compared to Ruxolitinib.

36. A method for preparing a pharmaceutical composition comprising performing (i) the method of any one of items 1 to 32, wherein the rAAV, the adenovirus, the lentivirus or the retrovirus comprises a therapeutic gene; and (ii) adding one more pharmaceutically acceptable excipient(s) to the prepared rAAV particles, adenovirus particles, lentivirus particles or retrovirus particles to thereby obtain a pharmaceutical composition.

37. A kit for use in cell culture comprising
(a) HEK293 cells; and
(b) an IFN inhibitor selected from the group consisting of Ruxolitinib, Tofacitinib, Baricitinib, Delgocitinib, Emapalumab and Fontolizumab and any structural analog thereof having IFN inhibitor activity, preferably the IFN inhibitor is Ruxolitinib and any structural analog thereof having at least 50% of the inhibitory activity for the Jak1 component in the IFN signaling pathway.

### EXAMPLES

### Example 1

### Materials and Methods

### Cell Culture, Fermentation

For this study, three derivates of HEK293 cells, all adapted to growth in suspension and cultivated in chemically defined serum-free media (FreeStyle™ F17 Expression Medium, Thermo Fisher, NY, USA), were used to produce rAAV8 vectors. The cell lines are all defined as HEK293 and treated in the same way. All used HEK293 cell lines have their origin in Frank Grahams Experiments in 1977, which enables the cells to produce rAAV. The cell line CRL-1573, further called CL1, was purchased from the American Type Culture Collection (ATCC) and adapted to serum free suspension culture. The cell line NRC HEK293SF-3F6, further called CL2, is commercially available from the National Research Council, Canada. The cell line HEK293 Pro10®, further called CL3, is commercially available from the company AskBio.

Every cell line was stored as separate cell bank partitioned in frozen stock-vials with 1.0×10⁶ cell/mL in FreeStyle™ F17 Expression Medium from Thermo Fisher with 7.5% DMSO to protect the cells from damage. For thawing the frozen stock-vials were placed from −120° C. to −80° C. overnight and thawed in a water bath to quickly transfer the cells into a 125 mL Corning® single use Spinner flask with pre-warmed Freestyle™ F17 Expression Medium. The cells were further propagated in Corning® disposable Spinner flasks for two weeks with regular passaging every second or third day for fresh media supply. The propagation process was performed in Thermo Fisher Heracell 150 Incubators with 37° C. and 5% carbon dioxide supply in a humidified atmosphere.

Adherent HEK293 cell line (Agilent Technologies CAT #240073, Lot #0006218516) was cultivated in T75 flasks in a total volume of 23 mL with DMEM media containing 2% FCS. The cultivation of the cells was performed in an incubator at +37° C. and 5% CO2. 1 mM Ruxolitnib stock solution was prepared by dissolving the solid white powder in DMSO according to the manufacturer's instructions before use and stored at −20° C. During the last passage before transfection Ruxolitinib was added to the cell culture at defined concentrations. Every condition was tested in duplicates. At transfection day a confluence range of 60 to 80% was achieved.

### Transfection

For transfection the HEK293 cells were transferred into 250 mL Sartorius SU bioreactors (Sartorius, Ambr® 250 modular Vessels, Mammalian), 10 L Eppendorf glass bioreactors (Eppendorf BioFlo®320) or 125 mL Corning® disposable shake flasks. All systems were adjusted to 37° C. and 5% carbon dioxide. The 250 mL bioreactors were constantly stirred at 492 rpm and the 10 L bioreactors were constantly stirred at 120 rpm and the pH was controlled with 0.5M sodium hydroxide solution. The shake flasks were placed in an Eppendorf New Brunswick S41i Incubator with 150 rpm agitation and a humidified atmosphere.

Transient transfection of HEK293 cells to produce rAAV8 capsids was performed with a three-plasmid system and carried out with polyethylenimine (PEI) (Merck KGaA, Darmstadt, Germany) following the supplier transfection protocol. The Adenovirus 5 Helper genes are delivered with one plasmid. The second plasmid was used to deliver Rep2Cap8 genes, which determine the rAAV Serotype, and the third plasmid codes for the potential therapeutic target gene, the human FIX sequence. The ratio between Helper, RepCap and transgene plasmids was 1:2:1.5. The plasmid vs. PEI ratio was 1:2.5. The transfection mix volume correlates to 10% of the final working volume and was composed of Freestyle™ F17 Expression Medium, plasmids and PEI. Before transfection the cells were passaged and adjusted to a target range between 3.0 and 5.0×10⁶ cells/mL.

Transient transfection of HEK293 to produce Lentivirus was performed with pALDI-Lenti system (Aldevron). This is a four-plasmid system including pALD-Lenti, pALD-VSV-G, pALD-GagPol and pALD-Rev suitable for the production of an HIV-1 derived viral vector. The transfection mix consisted of the four-plasmid system, DMEM media and PEI as transfection reagent. The ratio between the used plasmids was 1:1:1:1 and the plasmid DNA PEI ration was 1:3.

### Gene Set Enrichment Analysis

Gene set enrichment analysis was performed on the basis of the Hallmark gene sets available from UC San Diego and Broad Institute (https://www.sea-msigdb.org/gsea/msigdb/genesets.jsp?collection=H); see also Liberzon et al., Cell Syst. 23 (2015), 417-425.

### Next Generation Sequencing

For initial analysis of differences in the mRNA expression pattern of the three used HEK293 cell lines, Next Generation Sequencing was performed from 4 biological replicates of every cell line in a transfected and in a mock transfected condition at 5 different timepoints. The Mock transfection was performed without plasmids, only polyethylenimine (PEI) and Thermo Fisher Freestyle F17® Medium was added. The selected timepoints are 0 h (before Transfection), 4 h, 24 h, 48 h and 72 h post transfection. For every timepoint the same sampling procedure including 5.0×10⁶ cells/mL washed two times with PBS was performed. The cell pellets were analyzed with Illumina sequencing method. The output meta data contained the mRNA reads of the three HEK293 cell lines for all 5 timepoints and was used for exploring differences in transcriptomics.

### Results

The HEK293 cell lines used in the fermentative production produced different yields.

The results of the gene set enrichment analysis of rAAV transfected HEK293 cell lines showed differences in the activated cellular pathways.

Upon triple transfection with plasmids for rAAV production, the CL1 cell line showed an activation of the following genes:
(1) interferon_gamma response (0-48 h); and
(2) interferon_alpha_respone (0-48 h).

The activation of these genes was not observed in the CL3 cell line.

Thus, the genes activated by rAAV are significantly different in the CL3 cell line vs. the CL1 cell line.

In a further experiment the activation of cellular pathways in the CL1 cell line upon triple transfection for rAAV production in comparison to a mock transfection has been compared. It was found that the CL1 cell line upon triple transfection for rAAV production showed an activation of the following cellular pathways:
(i) TNFA_signaling_via_NFKB;
(ii) Interferon_Gamma_Response;
(iii) Interferon_Alpha_Response;
(iv) TGF_beta_Signaling;

Accordingly, this experiment confirmed the specific activation of particular genes in the CL1 cell line upon transfection with the plasmids necessary for rAAV production. The activation is rAAV specific since it could not be detected in the mock transfection.

### Example 2

### Inhibitor Treatment

Due to the fact that the CL1 cell line showed an activation of the interferon alpha and gamma response genes in contrast to the CL3 cell line, it was considered that the activation of said genes may contribute to the lower rAAV yield of the CL1 cell line as compared to the CL3 cell line.

For Inhibitor treatment the cells were incubated with a solubilized inhibitor for 2 h and up to 3 h before transfection. As Inhibitor the Janus associated Kinase (JAK) family Inhibitor Ruxolitinib, (Stemcell Technologies) was used and prepared according to the product instruction manual. After addition of the Inhibitor and transfection mix to the cell culture, the cells were further cultivated in batch mode for 48 h and up to 96 h.

### AAV8 ELISA

The commercially available enzyme-linked immunosorbent assay (ELISA; Progen AAV8 titration ELISA kit, cat. No. PRAAV8) uses a monoclonal antibody (ADK8) specific for a conformational epitope on assembled AAV8 capsids. This plate-immobilized antibody captures rAAV-8 particles from the specimen. Captured particles are then detected by the binding of biotinylated anti-AAV8 ADK8 since epitope targeted is repeatedly expressed on the assembled AAV8 capsid. Streptavidin peroxidase and a peroxidase substrate is then used for measuring bound anti-AAV8 and thus the concentration of AAV8 capsid. The color reaction was measured photometrically at 450 nm. The kit contains an rAAV2/8 particle preparation as calibration standard with a labelled rAAV8 particle concentration.

### P24 ELISA

The ELISA quantifies structural HIV1 p24 capsid protein amount in cell culture and supernatant samples. It is a quantitative sandwich ELISA method. For titering lentiviral expression HIV1 p24 ELISA Kit ab218268 was used. To discriminate between viral p24 produced by lentivirus and associated p24 produced by HEK293 cells Cell Biolabs Quick Titer™ Kit including LV-Origine/TR30021/Lot #134641F and the Cell Biolabs Reference Standard #310809 was used. The virus pulldown technology ensures detection of lentiviral associated p24 only.

Droplet Digital PCR (ddPCR)

For vector genome quantification, a Bio-Rad based droplet digital PCR method was used, applying fully-automated QX One System or automated QX 200 AutoDG system. This method provides absolute quantification of vector genome without the use of standard curves. Sample is partitioned into oil droplets and each droplet becomes an independent compartment for PCR reaction. Degeneration of capsids will take place in the initial phase of the PCR in the thermal cycler and DNA becomes accessible for amplification. Vector genome titer is then determined by using a droplet reader. Samples were treated with DNase I (NEB) to remove extraneous DNA sequences. Prior to the treatment samples were prediluted to increase the efficiency of the DNase I activity. After the droplet generation, PCR was performed with Bio-Rad ddPCR Supermix (no dUTP) and FIX-specific primers and probe:—Fwd 5′-GGC ATC TAC ACC AAA GTC TCC AG-3′ (SEQ ID NO: 1), Rev 5′-CAG CGA GCT CTA GGC ATG CT-3′ (SEQ ID NO: 2), probe 5′-6FAM-AGA CCA AGC TGA CCT GAT-MGBNFQ-3′ (SEQ ID NO: 3). Vector genome concentration was calculated by the appropriate Bio-Rad software.

### Results

### Shaker Flask Experiments

HEK293 cells (CL1) have been treated with different concentrations of IFN inhibitor Ruxolitinib. The final concentration of the IFN inhibitor is shown in Table 1 below:

| Shaker | Condition | Ruxolitinib conc. | |
|---|---|---|---|
| 1 | CL1 control | 0 | |
| 2 | CL1 control | 0 | |
| 3 | DMSO control | 0 | |
| 4 | DMSO control | 0 | |
| 5 | Concentration 1 | 1 | μM |
| 6 | Concentration 2 | 5 | μM |
| 7 | Concentration 3 | 10 | μM |
| 8 | Concentration 4 | 15 | μM |
| 9 | Concentration 5 | 20 | μM |
| 10 | Concentration 6 | 25 | μM |

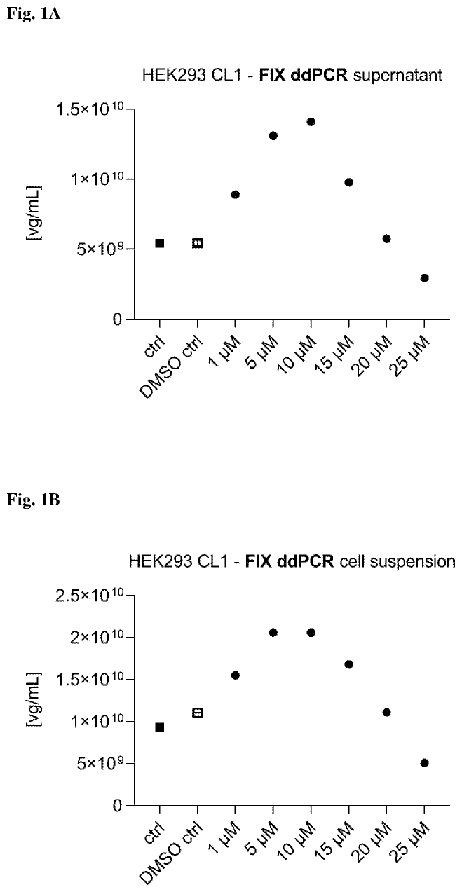

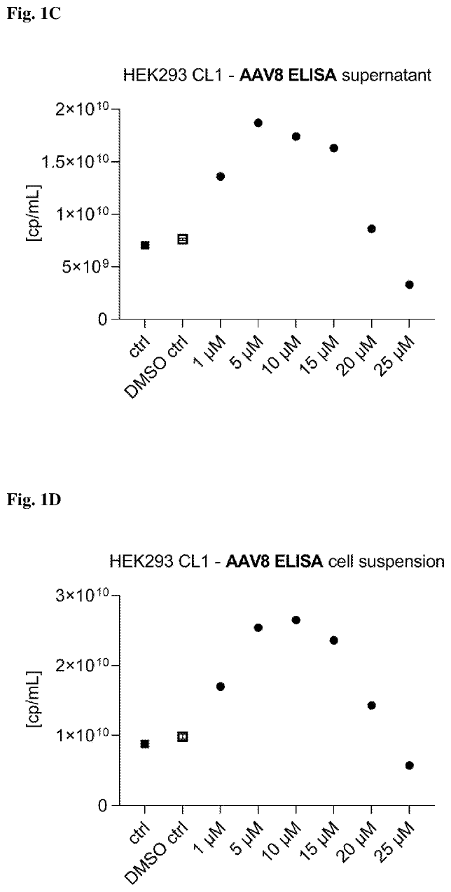

The yield of rAAV production as determined by vector genome droplet digital PCR (ddPCR) in the supernatant and cell culture, respectively is shown in FIGS. 1A and 1B, respectively. For the determination in the supernatant the vector genomes were exported into the media. For the determination in the cell culture, a sample of the cell culture was subjected to a freeze thaw cycle.

In the supernatant a concentration of 10 μM ruxolitinib resulted in an about 2.5-fold increase of the vector genome (vg) and an about 2-fold increase when determined in the cell suspension as compared to the negative control in the absence of the inhibitor. In the cell suspension an rAAV yield of about 2×10¹³ vg/L cell culture at an inhibitor concentration of 5 μM and 10 μM ruxolitinib, respectively, has been observed.

The yield of rAAV production as determined by capsid particle in the supernatant and cell culture is shown in FIGS. 1C and 1D, respectively. Similar results as compared to the ddPCR determination have been observed.

It can be seen that by the addition of IFN inhibitor a significant increase in the yield of rAAV from these Inhibitor treated CL1 cells as compared to the yield in the absence of IFN inhibitor is observed. The optimal concentration of IFN inhibitor was 5 μM and 10 PM. The increase as determined in cell suspension was about 3-fold as compared to the rAAV yield in the absence of the inhibitor. For concentrations higher than 10 μM a reduction of the effect has been observed.

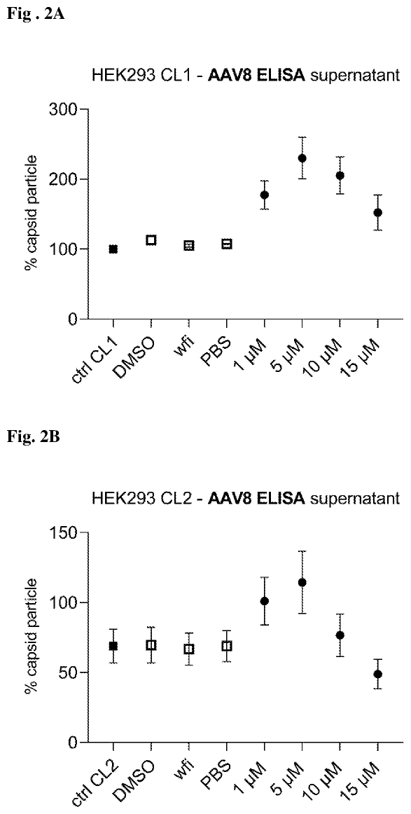

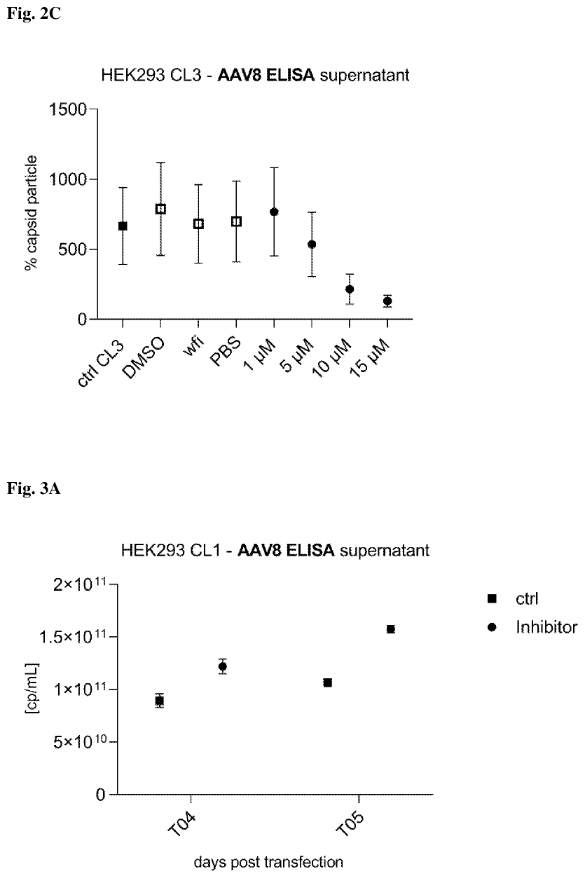

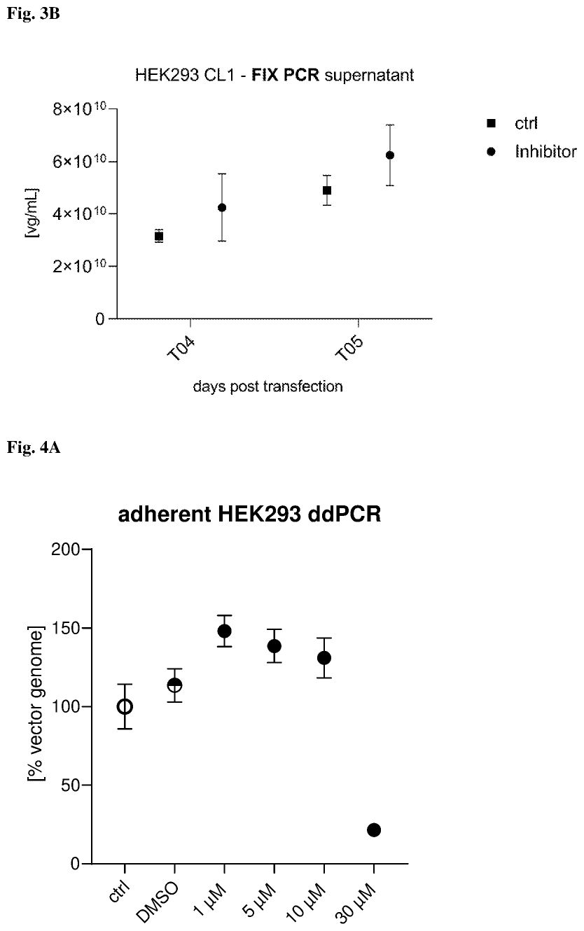

In a separate set of experiments the rAAV yield of three different cell lines (CL1, CL2 and CL3) in the absence (ctrl) or presence of Inhibitor was compared. The rAAV yield of CL1 ctrl was used as basis (100%) for comparing the yields among the different cells lines used, see FIGS. 2(A), 2(B) and 2(C). In order to rule out any effects of solvents for the Inhibitor, experiments were also conducted in the presence of the respective solvent without inhibitor (DMSO, water for injection (wfi), PBS). The results determined by rAAV8 ELISA assay in the supernatants is shown in FIG. 2. FIG. 2(A) relates to the CL1 cell line; FIG. 2(B) to the CL2 cell line and FIG. 3(C) to the CL3 cell line.

It can be seen that for each of the different HEK293 cell lines an increase of rAAV8 yield could be obtained by adding an IFN inhibitor to the culture medium. The effect apparently is higher the lower rAAV production of the HEK293 cell is in the absence of the IFN inhibitor.

The present invention is therefore universally applicable to HEK293 cells.

### 10 L Bioreactor Experiments

To test the effect of IFN inhibitor on the rAAV yield in large-scale bioreactor fermentation of HEK293 cells, 10 L bioreactors have been used. The results are shown in FIG. 3A (determination by capsid particlerAAV8 ELISA) and FIG. 3B (determination by ddPCR). The ddPCR results confirm an improvement with Interferon Inhibitor (approximately 1.3-fold improvement determined in the supernatant). rAAV8 capsid particle ELISA results showed an even higher improvement of about 1.5 fold in supernatant. Experiments for the determination of rAAV8 capsid particles by ELISA showed an even higher improvement of approximately 1.5-fold increase in the Inhibitor treated experiment as compared to the negative control in the absence of the inhibitor.

Results obtained from 3 L Single Use bioreactors showed a highly comparable performance compared to 10 L bioreactors.

### Adherent HEK293 Experiments in T-Flasks

Adherent HEK293 cells have been treated with different concentrations of IFN inhibitor Ruxolitinib. The final concentration of the IFN inhibitor is shown in Table 2 below:

| Shaker | Condition | Ruxolitinib conc. | |
|---|---|---|---|
| 1 | Adherent control (ctrl) | 0 | |
| 2 | DMSO control | 0 | |
| 5 | Concentration 1 | 1 | μM |
| 6 | Concentration 2 | 5 | μM |
| 7 | Concentration 3 | 10 | μM |
| 8 | Concentration 4 | 30 | μM |

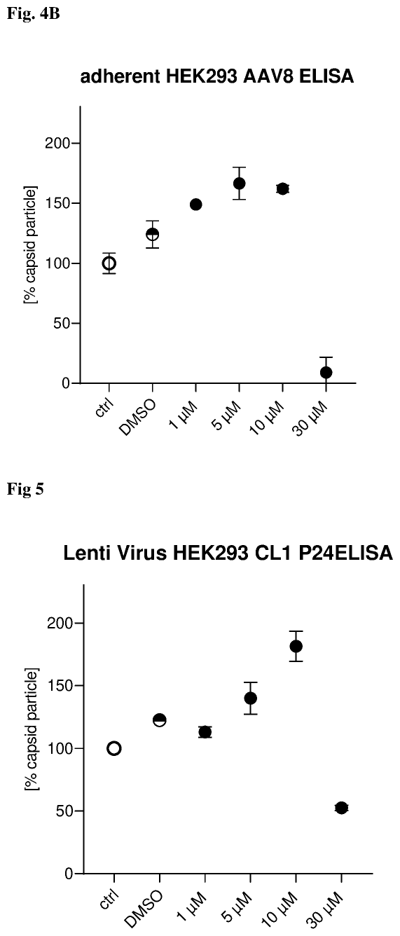

The yield of rAAV production as determined by vector genome droplet digital PCR (ddPCR) in the supernatant is shown in FIG. 4A. For the determination of the capsid particle concentration AAV8 ELISA was used as shown in FIG. 4B. The statistical mean of the control condition was calculated as 100%.

The ddPCR results of the supernatant samples show a yield increase of 48% with the inhibitor concentration of 1 μM compared to the control condition. Concentrations of 5 μM and 10 μM inhibitor show a vg yield increase of 38.5 and 31%, respectively.

Capsid particle ELISA results for supernatant samples of adherent HEK293 cells show the highest yield increase of 66.5% compared to the control condition with an inhibitor concentration of 5 μM. A concentration of 10 μM show 62% titer increase and a concentration of 1 μM show and improvement of 49% capsid particles in the supernatant.

All experiments were performed in duplicates and showed a high comparability. Specific doses of 1 up to 5 μM lead to a yield increase in ddPCR vg and ELISA cp detection. A positive effect of the inhibitor with adherent HEK293 cells can be observed.

Suspension HEK293 (CL1) Experiments with Lentivirus

The suspension HEK293 (CL1) experiments with a four-plasmid lentiviral transfection were performed in shake flasks with the same conditions as shown in Table 2. Lentiviral cp titer was determined by a p24 sandwich ELISA shown in FIG. 5. The statistical mean of the control condition was evaluated as 100%.

The maximum cp titer increase of 81.5% in suspension HEK293 transfected with lentiviral plasmids is shown with 10 μM inhibitor concentration compared to the untreated control condition. The 5 μM inhibitor concentration show a yield increase of 40% compared to the control.

The positive effect of IFN pathway inhibition with HEK293 cells (CL1) transfected with lentiviral plasmids was confirmed.

1. A method for the fermentative production of an adeno-associated virus (AAV), an adenovirus, a lentivirus or a retrovirus comprising the steps of:
Step 1: culturing the rAAV, the adenovirus, the lentivirus or the retrovirus in a HEK293 cell in a culture medium comprising an amount of an interferon (IFN) inhibitor over an incubation period; and
Step 2: recovering the rAAV, the adenovirus, the lentivirus or the retrovirus from the cell culture.

2. The method of claim 1, wherein the IFN inhibitor is not selected from niacin and/or niacinamide.

3. The method of claim 1 or 2, wherein the yield of the rAAV, the adenovirus, the lentivirus or the retrovirus over an incubation period of at least 96 hours is increased by at least 10%, preferably by at least 50% compared to the yield of the rAAV, the adenovirus, the lentivirus or the retrovirus in the absence of the IFN inhibitor, more preferably by at least 100%, even more preferred by at least 200%.

4. The method of any of claims 1 to 3, wherein in the presence of the IFN inhibitor from the culture medium the HEK293 cell produces an rAAV, lentivirus or retrovirus yield of at least about 1×10⁹ purified vector genome-containing particles per milliliter of cell culture (vg/mL), preferably at least about 5×10⁹, more preferably at least 1×10¹⁰, more preferred of at least 5×10¹⁰, and most preferred at least 1×10¹¹, 1×10¹², 1×10¹³, 1×10¹⁴ or 5×10¹⁴ more vector genome-containing particles per milliliter of cell culture.

5. The method of any one of claims 1 to 4, wherein the HEK293 cell is characterized upon rAAV transfection by an activation of at least one of the following genes:
(1) interferon_gamma response; and/or
(2) interferon_alpha_response.

6. The method of claim 5, wherein the HEK293 cell is characterized upon rAAV transfection by an activation of at least interferon_gamma response and interferon_alpha_response.

7. The method of any one of claims 1 to 6, wherein the HEK293 cell line is characterized upon rAAV transfection by an activation of at least one of the following genes:
(i) TNFA_signaling_via_NFKB;
(ii) Interferon_Gamma_Response;
(iii) Interferon_Alpha_Response;
(iv) TGF_beta_Signaling; and/or
(v) IL6_JAK_STAT3_Signaling.

8. The method of claim 7, wherein the at least 2 activated genes are selected from the group consisting of TNFA_signaling_via_NFKB, interferon_gamma response, interferon_alpha_response, TGF_beta_Signaling, and IL6_JAK_STAT3_Signaling, more preferably the at least 2 activated genes are selected from the group consisting of interferon_gamma response, interferon_alpha_response and IL6_JAK_STAT3_Signaling.

9. The method of claim 7 or 8, wherein the HEK293 cell shows an activation of at least 2, at least three, at least four pathways, preferably each of the five pathways are activated.

10. The method according to any one of claims 1 to 9, wherein the HEK293 cell is in the presence of the IFN inhibitor from the culture medium characterized by an AAV yield of at least 1×10¹⁰, more preferred of at least 5×10¹⁰, and most preferred at least 1×10¹¹, 1×10¹², 1×10¹³, 1×10¹⁴ or 5×10¹⁴ or more capsid particles per milliliter culture.

11. The method according to any one of claims 1 to 10, wherein the HEK293 cell is an adherent cell line.

12. The method according to any one of claims 1 to 10, wherein the HEK293 cell is a suspension cell line, or adapted to grow in suspension.

13. The method according to any one of claims 1 to 12, wherein the HEK293 cell is selected from the group consisting of CRL-1573 and NRC (HEK293SF-3F6).

14. The method according to any one of claims 1 to 13, wherein Step 1 is carried out in a petri dish, a shaker flask, a rocking bag or a roller bottle.

15. The method according to any one of claims 1 to 13, wherein Step 1 is carried out in a bioreactor, preferably the bioreactor comprises a volume of at least 10 L.

16. The method according to any one of claims 1 to 15, wherein the rAAV is selected from any AAV serotype selected from AAV1, AAV2, AAV3, AAV4, AAV5, AAV6, AAV7, AAV8, AAV9, AAV10, AAV11, AAV12 or AAV13, chimeric and hybrid variants thereof, preferably the AAV serotype is selected from AAV5, AAV6, AAV8, and AAV9, more preferably AAV8.

17. The method according to any one of claims 1 to 15, wherein the lentivirus is derived from a human immunodeficiency virus (HIV), preferably the lentivirus is derived from HIV-1.

18. The method according to claim 17, wherein the lentivirus vector is obtained by transfection of the HEK293 cell with at least one packaging plasmid, preferably in combination with an envelope plasmid derived from vesicular stomatitis virus (VSV), more preferably the envelope plasmid is derived from VSV-G.

19. The method according to any one of claims 1 to 15, wherein the retrovirus is murine leukemia virus (MuLV).

20. The method according to any one of claims 1 to 19, wherein the IFN inhibitor is a molecule which decreases the activity of the IFN induction and/or signalling cascades.

21. The method according to claim 20, wherein the IFN inhibitor targets the JAK/STAT signaling pathway.

22. The method according to claim 21, wherein the IFN inhibitor targets JAK family kinases.

23. The method according to any one of claims 20 to 22, wherein the IFN inhibitor is selected from the group consisting of Ruxolitinib, Tofacitinib, Baricitinib, Delgocitinib, Emapalumab and Fontolizumab, and any structural analog thereof having IFN inhibitor activity, preferably the IFN inhibitor is Ruxolitinib and any structural analog thereof having at least 50% of the inhibitory activity for the JAK family kinases in the IFN signaling pathway as compared to Ruxolitinib.

24. The method according to any one of claims 1 to 23, wherein the final concentration of the IFN inhibitor in the medium is in the range from about 0.0001 to 50 mM, preferably about 1 to about 100 μM, more preferably about 0.5 to about 25 μM, even more preferred the final concentration is about 1 μM, about 5 μM, about 10 μM, about 15 μM, about 20 μM or about 25 μM.

25. The method according to any one of claims 1 to 24, wherein the culture medium is a chemically defined medium.

26. The method according to any one of claims 1 to 25 comprising the following steps:
(a) culturing HEK 293 cells in a culture medium containing an amount of an IFN inhibitor over a first incubation period;
(b1) transfecting the HEK293 cells with three plasmids necessary for rAAV production, preferably with (i) an adenovirus helper plasmid, (ii) a packaging rep-cap plasmid and (iii) a recombinant rAAV plasmid comprising a transgene; or
(b2) transfecting the HEK293 cells with two plasmids necessary for rAAV production, preferably with (i) a plasmid comprising the adenovirus helper genes and the rep-cap genes and (ii) a recombinant rAAV plasmid comprising a transgene;
(c) culturing of the transfected HEK293 cells over a second incubation period; and
(d) recovering the AAV particles.

27. The method of claim 26, wherein the HEK293 cells are cultured with the IFN inhibitor in step (a) over a period of about 1 hour to about 12 hours, preferably 1 to 6, more preferably 1 to 3 hours before step (b) is performed.

28. The method of claim 26 or 27, wherein the (i) adenovirus helper plasmid comprises one or more of the adenovirus genes selected from the group consisting of E1A, E1B, E2A, E40RF6 and VA; and/or (ii) the packaging rep-cap plasmid comprises a Rep2 gene and a Cap8 gene and/or (iii) the AAV plasmid comprises a 5′ AAV ITR and a 3′ AAV ITR from the same AAV serotype, preferably from AAV2.

29. The method of any one of claims 26 to 28, wherein the transfection step is carried out using polyethyleneimine or FectoVIR™-AAV as transfection reagent.

30. The method of any one of claims 26 to 29, wherein the second incubation period is at least 24 hours, preferably at least 48 hours, more preferably at least 72 hours, most preferred at least 96 hours.

31. The method of any one of claims 26 to 30, wherein insofar the rAAV is secreted from the HEK293 cells step (d) includes collecting of the supernatant of the culture medium and purifying the rAAV particles from the supernatant.

32. The method of any one of claims 26 to 31, wherein insofar the rAAV is not secreted from the HEK293 cells step (d) includes a step of disrupting the HEK293 cells to obtain the rAAV particles and subsequent purifying of the rAAV particles.

33. A method for the fermentative production of an adeno-associated virus (AAV), a lentivirus or a retrovirus comprising the steps of:
Step 1: performing a transcriptomic analysis on HEK293 cells transfected with an rAAV, an adenovirus, a lentivirus or a retrovirus and selecting the HEK293 cell if the transcription of at least one of the following genes is enriched compared to a negative control:
(i) TNFA_signaling_via_NFKB;
(ii) Interferon_Gamma_Response;
(iii) Interferon_Alpha_Response;
(iv) TGF_beta_Signaling; and/or
(v) IL6_JAK_STAT3_Signaling,
preferably if the transcription of
(1) interferon_gamma response; and/or
(2) interferon_alpha_respone
is enriched;
Step 2: culturing the rAAV, the adenovirus, the lentivirus or the retrovirus in the selected HEK293 cell in a culture medium comprising an amount of an interferon (IFN) inhibitor over an incubation period; and
Step 3: recovering the rAAV, the adenovirus, the lentivirus or the retrovirus from the cell culture.

34. Use of an IFN-inhibitor for increasing the yield of rAAV, adenovirus, Lentivirus or retrovirus in a culture of HEK293 cells transfected or infected with the rAAV, the adenovirus, the lentivirus or the retrovirus.

35. Use of claim 34, wherein the IFN inhibitor is not selected from niacin and/or niacinamide.

36. Use of claim 34 or 35, wherein the HEK293 cell is characterized upon transfection or infection with the rAAV, the adenovirus, the lentivirus or the retrovirus by an activation of at least one of the following cellular pathways:
(1) interferon_gamma response; and/or
(2) interferon_alpha_response.

37. Use according to claim 36, wherein the IFN inhibitor is selected from the group consisting of Ruxolitinib, Tofacitinib, Baricitinib, Delgocitinib, Emapalumab and Fontolizumab, and any structural analog thereof having IFN inhibitor activity, preferably the IFN inhibitor is Ruxolitinib and any structural analog thereof having at least 50% of the inhibitory activity for the JAK family kinases in the IFN signaling pathway as compared to Ruxolitinib.

38. A method for preparing a pharmaceutical composition comprising performing (i) the method of any one of claims 1 to 34, wherein the rAAV, the adenovirus, the Lentivirus or the retrovirus comprises a therapeutic gene; and (ii) adding one more pharmaceutically acceptable excipient(s) to the prepared rAAV particles, adenovirus particles, Lentivirus particles or retrovirus particles to thereby obtain a pharmaceutical composition.

39. A kit for use in cell culture comprising
(a) HEK293 cells; and
(b) an IFN inhibitor selected from the group consisting of Ruxolitinib, Tofacitinib, Baricitinib, Delgocitinib, Emapalumab and Fontolizumab and any structural analog thereof having IFN inhibitor activity, preferably the IFN inhibitor is Ruxolitinib and any structural analog thereof having at least 50% of the inhibitory activity for the JAK family kinases in the IFN signaling pathway.
